(19) United States
(12) Patent Application Publication
(10) Pub. No.: US 2022/0330806 A1
(43) Pub. Date: Oct. 20, 2022

(54) Rigid endotracheal intubation stylet with extended, angulated, tapered, and pliable tip

(71) Applicant: Samantha Reeder, Gilbert, AZ (US)

(72) Inventor: Samantha Reeder, Gilbert, AZ (US)

(21) Appl. No.: 17/698,978

(22) Filed: Mar. 18, 2022

Related U.S. Application Data
(60) Provisional application No. 63/258,169, filed on Apr. 16, 2021.

(51) Int. Cl. A61B 1/267; A61M 16/04; A61M 25/01

(52) U.S. Cl. CPC A61B 1/267; A61M 16/049; A61M 25/0102

(57) ABSTRACT

A rigid intubation stylet with an extended tip for use during video laryngoscopic intubations. This tip is hyper angulated from the end of the stylet back towards the stylet body. The shape of the tip is conical and tapers down away from the body of the stylet. The tip is more pliable than the rigid stylet body. The stylet body is longer than traditional rigid stylets to allow the tip to extend past an endotracheal tube during endotracheal intubation.

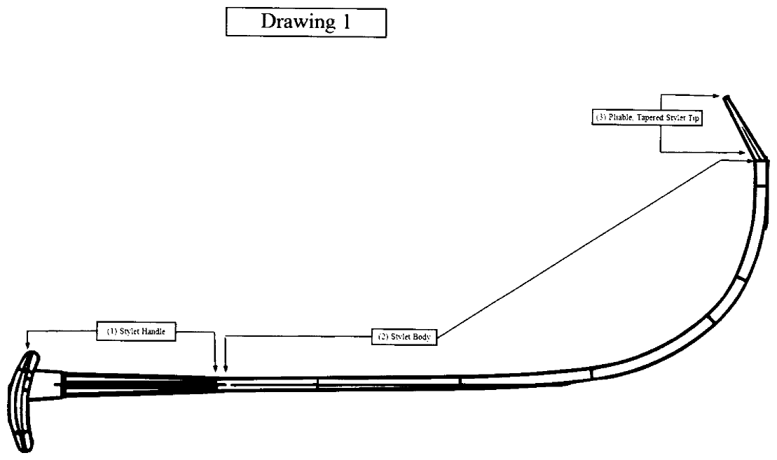

### BACKGROUND

This invention relates to rigid stylets for patient airway management by way of video-directed endotracheal intubations. Video endotracheal intubations are performed during procedural and emergent airway management. The goal of this procedure is to establish a definitive airway for a patient to breath by placing one end of a hollow tube into the trachea and past the glottis and the other external to the patient. When performing an endotracheal intubation via video laryngoscopy, the medical provider does not always have a clear path for the endotracheal tube to enter the airway. To accomplish this goal, a rigid stylet is inserted inside an endotracheal tube and maneuvered inside a patient's upper airway to position the endotracheal tube to pass through the vocal cords. Once the endotracheal tube is past the vocal cords, the provider removes the rigid stylet, establishing a definitive airway. Stylets currently in use should remain inside the distal portion of the endotracheal tube throughout the procedure and never make contact with the patient's vocal cords.

However, a patient's airway could be obscured or mispositioned in relation to the provider's view, therefore making it difficult or impossible to maneuver the endotracheal tube into the patient's airway. Under these circumstances, the endotracheal tube is effectively blocked from passing the vocal cords and entering the trachea to establish the airway. The angle of entry for the tube can become difficult to reach or the actual opening between the vocal cords too small to penetrate. Current rigid stylets are not designed to make contact with the patient due to the stylet's lack of protrusion from the endotracheal tube. Additionally, if current stylets were to protrude past the endotracheal tube, their composition would result in damage to the trachea. This limitation decreases the success of the procedure and delays critical patient care.

### SUMMARY OF INVENTION

It is the object of this invention to solve the problem of difficult video endotracheal tube intubation by providing a rigid stylet with a flexible and protruding tip. The rigid portion of the stylet is inserted into the endotracheal tube, but the tip of this stylet protrudes from the tube. The extended tip is pliable so it may pass through and make contact with the vocal cords without harming the trachea. Additionally, angulation of the tip decreases the amount of manipulation needed to direct the endotracheal tube because the tip is in an anatomically beneficial position i.e. it better aligns with the human airway. The tapered, pliable tip acts to dilate the vocal cords and provide an accessible opening for the endotracheal tube. Once the stylet's tip is past the vocal cords, the endotracheal tube is advanced along the elongated tip of the stylet directly into the trachea to establish the airway.

### DETAILED DESCRIPTION

The purpose of this invention is to increase first-pass success rate in video endotracheal tube intubations by introducing a new rigid stylet with unique, protruding tip. In performing the procedure with this new stylet, the healthcare provider will insert the new rigid stylet into the lumen of an endotracheal tube so that the tip of the stylet protrudes roughly 2 to 3 centimeters past the end of the tube. The pliable tip is the only portion of the stylet that should protrude from endotracheal tube opposite the handle side of the stylet. Once assembled in this fashion, the healthcare provider will hold the tube and stylet so that the handle is external to the patient and the tip is entering the oropharynx. Posterior to the epiglottis, the tube and stylet navigate toward the patient's vocal cords. The combined rigidity of the stylet and the pliability of the tip grants the medical provider maneuverability in an airway while minimizing concern of perforating tracheal walls. Additionally, because of its composition and protrusion from the tube, the tip will act as a hyper angulated director that is thinner than the endotracheal tube to be able to turn anteriorly and slip through the vocal cords to easily direct the tube into the trachea. The pliable, tapered tip of the stylet is then used to pass the vocal cords, dilating the cords as the tip and tube are advanced. Once the tip is successfully past the vocal cords, the endotracheal tube advances into the trachea to be secured and the stylet is backed out of the tube by the handle.

### DESCRIPTION OF DRAWING

The attached drawing in this application titled “Drawing 1” is a drawing of the pliable, tapered tip that extends past the end of a loaded endotracheal tube.

1. A rigid intubation stylet comprising: a tip that protrudes past the end of an endotracheal tube when the tube is loaded onto the stylet. The tip protrudes past the endotracheal tube during an endotracheal intubation procedure until the tube is advanced off the stylet.

2. A rigid intubation stylet according to claim 1, wherein the tip is more pliable than the body of the stylet.

3. A rigid intubation stylet according to claim 1, wherein the tip is hyper-angulated back towards the handle of the stylet.

4. A rigid intubation stylet according to claim 1, wherein the tip narrows from the body of the stylet to the termination of the tip.
